(19) United States
(12) Patent Application Publication
(10) Pub. No.: US 2023/0052833 A1
(43) Pub. Date: Feb. 16, 2023

(54) OIL-BASED PARTICLES AND EXTERNAL COMPOSITION

(71) Applicant: SHISEIDO COMPANY, LTD., Tokyo (JP)

(72) Inventors: Takahiro KATORI, Tokyo (JP); Yurika WATANABE, Tokyo (JP); Tsukasa WATANABE, Tokyo (JP)

(21) Appl. No.: 17/757,901

(22) PCT Filed: Dec. 17, 2020
(86) PCT No.: PCT/JP2020/047151

§ 371 Date: Jun. 23, 2022

(30) Foreign Application Priority Data
Dec. 27, 2019 (JP) 2019-239664

(51) Int. Cl. A61K 8/06; A61K 8/92

(52) U.S. Cl. CPC A61K 8/068; A61K 8/92; A61K 2800/412; A61K 2800/10

(57) ABSTRACT

Oil-based particles have an average particle size of 0.05 mm to 10 mm, and include: (A) polar oil; and (B) an oily gelling agent having a melting point of 60° C. or more.

### TECHNICAL FIELD

The present invention relates to oil-based particles and an external composition.

### BACKGROUND ART

Cosmetics such as creams, emulsions, liquid foundations, hair conditioners, and the like, are required to contain water-soluble ingredients and oils at the same time, such as water and moisturizers. Emulsification is a state in which one of two liquids that do not mix with each other, such as oil and water, becomes fine particles and is dispersed in the other. When the dispersed emulsified particles are large, water and oil are easily separated. Therefore, conventionally, a method for making the emulsified particles as small as possible (1 to 50 μm) has been developed.

When such particles are large, the range of substances that can be contained in the particles and their contents is widened, and there are many advantages such as improving a feeling of use at the time of application. However, when the emulsified particles are enlarged, separation, aggregation, coalescence, and the like occur, and stability as emulsified particles is not obtained. Accordingly, a completely different structure from the emulsified particles produced by using a surfactant, that is, a lipid shell, which is an oil-based particle larger (10 mm) than conventional emulsified particles, in which the outside of the particles is covered with a shell of solid oil, has been developed (PTL 1).

### CITATION LIST

### Patent Literature

### [PTL 1]

Japanese Laid-Open Patent Publication No. 2012-20965

### [PTL 2]

Japanese Laid-Open Patent Publication No. 2005-36001

### SUMMARY OF INVENTION

### Technical Problem

However, conventional lipid shells were unable to contain a large amount of polar oil, which is a highly polar oil. Accordingly, one aspect of the present invention is to provide oil-based particles that can contain a large amount of polar oil.

### Solution to Problem

As a result of intensive research to solve the above problem by the inventors of the present invention, one aspect of the present invention is: oil-based particles having an average particle size of 0.05 mm to 10 mm, the oil-based particles including: (A) polar oil; and (B) an oily gelling agent having a melting point of 60° C. or more.

### Advantageous Effects of Invention

According to one aspect of the present invention, oil-based particles that can contain a large amount of polar oil, can be provided.

### DESCRIPTION OF EMBODIMENTS

One aspect of the present invention relates to oil-based particles having an average particle size of 0.05 to 10 mm, the oil-based particles including: (A) polar oil; and (B) an oily gelling agent having a melting point of 60° C. or more. Such oil-based particles can enhance the stability of the lipid shell even when a certain amount of polar oil is contained in a high content. Hereinafter, one aspect of the present invention will be described in detail, but embodiments of the present invention are not limited thereto.

### <Oil-Based Particle>

In the oil-based particles according to one aspect of the present invention, the average particle size is 0.05 to 10 mm, and a shell, which is solid oil, covers liquid oil. The solid oil is mainly present at the interface between the oil phase and the aqueous phase, but may be partially present inside the oil-based particle together with other oily ingredients. The liquid oil may be partially present at the interface between the oil phase and the aqueous phase. With such a configuration, when applied to the skin as an external agent, the inhibition of penetration of water into the skin by the oil phase ingredients is prevented. Further, because water-soluble thickener is present in the aqueous phase, the oil phase can be stably dispersed in the aqueous phase without adding a surfactant.

### <(A) Polar Oil>

(A) The polar oil is not particularly limited as long as it is usually used in cosmetics, pharmaceuticals, and foods. As an index indicating degree of polarity of organic compounds, IOB value (Inorganic/Organic Balance (inorganic value/organic value)) indicating ratio of inorganic value to organic value is used. Regarding the “inorganic value” and the “organic value” in the IOB value, the “inorganic value” and the “organic value” are set according to various atoms or functional groups. For example, the “organic value” is 20 for one carbon atom in a molecule, and the “inorganic value” is 100 for one hydroxyl group in a molecule. The IOB value of the organic compound may be calculated by integrating the “inorganic value” and the “organic value” described above of all the atoms and functional groups in the organic compound (see, for example, Fujita, Vol. 11, No. 10, pp. 719-725, 1957). The IOB value of the polar oil according to one aspect of the present invention is preferably, but not limited to, 0.05 to 0.80.

Examples of such polar oil include oils having an ester group and a polyoxyalkylene group continuously, such as cetyl octanate having an ester and a polyoxyalkylene group, and vegetable oils.

As such polar oil, (A) the polar oil according to one aspect of the present invention may contain polar oil having an ultraviolet absorbing function (hereinafter, may also be referred to as ultraviolet absorbing polar oil). The ultraviolet absorbing polar oil contained in (A) the polar oil is not particularly limited. Examples of the ultraviolet absorbing polar oil include, benzoic acid derivatives, salicylic acid derivatives, cinnamic acid derivatives, dibenzoylmethane derivatives, β,β-diphenylacrylate derivatives, benzophenone derivatives, benzylidene camphor derivatives, phenylbenzimidazole derivatives, triazine derivatives, phenylbenzotriazole derivatives, anthranilic acid derivatives, imidazoline derivatives, benzalmalonate derivatives, 4,4-diarylbutadiene derivatives, and the like. Specifically, it is as follows.

Examples of the benzoic acid derivatives include ethyl p-aminobenzoate (PABA), ethyl-dihydroxypropyl PABA, ethylhexyl-dimethyl PABA, glyceryl PABA, PEG-25-PABA, diethylamino hydroxybenzoyl hexyl benzoate, and the like.

Examples of the salicylic acid derivatives include homosalate, ethylhexyl salicylate (octyl salicylate), dipropylene glycol salicylate, TEA salicylate, and the like.

Examples of the cinnamic acid derivatives include octyl methoxycinnamate or ethylhexyl methoxycinnamate, isopropyl methoxycinnamate, isoamyl methoxycinnamate, cinoxate, DEA methoxycinnamate, diisopropyl methylcinnamic acid, glyceryl-ethylhexanoate-dimethoxycinnamate, di-(2-ethylhexyl)-4′-methoxybenzal malonate, and the like.

Examples of the dibenzoylmethane derivatives include butyl methoxybenzoylmethane (4-tert-butyl-4′-methoxydibenzoylmethane), and the like.

Examples of the β,β-diphenylacrylate derivatives include octocrylene, and the like.

Examples of the benzophenone derivatives include benzophenone-1, benzophenone-2, benzophenone-3 or oxybenzone, benzophenon-4, benzophenon-5, benzophenon-6, benzophenon-8, benzophenon-9, benzophenon-12, and the like.

Examples of the triazine derivatives include anisotriazine (bis-ethylhexyloxyphenol methoxyphenyltriazine), ethylhexyltriazone, diethylhexylbutamide triazone, 2,4,6-tris(diisobutyl-4′-aminobenzalmalonate)-s-triazine, and the like.

Examples of the phenylbenzotriazole derivatives include drometrizol trisiloxane, methylenebis(benzotriazolyltetramethylbutylphenol), and the like.

Examples of the anthranilic acid derivatives include menthyl anthranilate, and the like.

Examples of the imidazoline derivatives include ethylhexyl dimethoxybenzylidene dioxoimidazolidine propionate, and the like.

Examples of the benzalmalonate derivatives include a polyorganosiloxane having a benzalmalonate functional group, and the like.

Examples of the 4,4-diarylbutadiene derivatives include 1,1-dicarboxy(2,2′-dimethylpropyl)-4,4-diphenylbutadiene, and the like.

(A) The polar oil according to one aspect of the present invention may contain the ultraviolet absorbing polar oil described above alone or in combination of two or more. For example, as the ultraviolet absorbing polar oil, ethylhexyl methoxycinnamate, bis-ethylhexyloxyphenol methoxyphenyl triazine, oxybenzone-3, diethylamino hydroxybenzoyl hexyl benzoate, homosalate, ethylhexyl salicylate (octyl salicylate), butyl methoxydibenzoylmethane, octocrylene, anisotriazine (bis-ethylhexyloxyphenol methoxyphenyltriazine), and ethylhexyltriazone may be contained in combination. As described above, it is preferable to use a combination of a plurality of ultraviolet absorbing polar oils including the derivatives derived from different precursor compounds. Accordingly, a wide range ultraviolet protection effect can be provided. In particular, it is preferable that ethylhexyl methoxycinnamate, bis-ethylhexyloxyphenol methoxyphenyl triazine, and diethylamino hydroxybenzoyl hexyl benzoate are contained alone or in combination of two or more.

The oil-based particles according to one aspect of the present invention in which (A) the polar oil contains the ultraviolet absorbing polar oil, are preferably used for sunscreen cosmetics and quasi-drugs.

As described above, when (A) the polar oil contains the ultraviolet absorbing polar oil, either only the ultraviolet absorbing polar oil may be contained, or the ultraviolet absorbing polar oil and a polar oil that does not have an ultraviolet absorbing function may be contained in combination. Examples of the polar oil that does not have an ultraviolet absorbing function include ester oils, polyalkylene oxide derivative oils, and the like. Specifically, it is as follows.

Examples of the ester oils include isononyl isononanoate, isopropyl myristate, cetyl 2-ethylhexanoate, octyldodecyl myristate, isopropyl palmitate, butyl stearate, hexyl laurate, myristyl myristate, decyl oleate, hexyldecyl dimethyloctanoate, cetyl lactate, myristyl lactate, lanolin acetate, isocetyl stearate, isocetyl isostearate, cholesteryl 12-hydroxystearate, ethylene glycol di(2-ethylhexanoate), dipentaerythritol fatty acid ester, N-alkyl glycol monoisostearate, neopentyl glycol dicaprate, diisostearyl malate, glyceryl di-2-heptylundecanoate, trimethylolpropane tris(2-ethylhexanoate), trimethylolpropane triisostearate, pentaerythritol tetra(2-ethylhexanoate), glyceryl tri(2-ethylhexanoate) (triethylhexanoin), trimethylolpropane triisostearate, cetyl 2-ethylhexanoate, 2-ethylhexylpalmitate, diethylhexyl 2,6-naphthalate, benzoic acid (12 to 15 carbon atoms) alkyl, cetearyl isononanoate, tri (caprylic acid/capric acid) glycerin, (dicaprylic acid/capric acid) butylene glycol, glyceryl trimyristate, tri 2-heptylundecanoate glyceride, castic oil fatty acid methyl ester, oleyl oleate, cetosteryl alcohol acetoglyceride, 2-heptylundecyl palmitate, diisobutyl adipate, 2-octyldodecyl N-lauroyl-L-glutamate, di(2-heptylundecyl) adipate, ethyllaurate, di(2-ethylhexyl) sebacate, 2-hexyldecyl myristate, 2-hexyldecyl palmitate, 2-hexyldecyl adipate, diisopropyl sebacate, di-2-ethylhexyl succinate, ethyl acetate, butyl acetate, amyl acetate, triethyl citrate, tripropylene glycol dipivalate, and the like. In particular, diisopropyl sebacate is preferably used.

Examples of the polyalkylene oxide derivative oils include diethylene glycol monopropyl ether, polyoxyethylene polyoxypropylene pentaerythritol ether, polyoxypropylene butyl ether, and the like.

As (A) the polar oil according to one aspect of the present invention, the ester oil and the polyalkylene oxide derivative oil described above may be contained either alone or in combination of two or more. When the polar oil containing the ester oil is used in combination with the ultraviolet absorbing polar oil, the dissolution of the solid ultraviolet absorbing polar oil is promoted. For example, it is preferable that each of ethylhexyl methoxycinnamate, bis-ethylhexyloxyphenol methoxyphenyl triazine, and diethylamino hydroxybenzoyl hexyl benzoate is contained alone or in combination of two or more, in combination with diisopropyl sebacate.

When a large amount of polar oil is contained in the lipid shell particle, a tactile feeling when used is improved. When a large amount of the ultraviolet absorbing polar oil is contained in the lipid shell particle, the function as a cosmetic is enhanced. A product containing the lipid shell, which has a comfortable tactile feeling when applied as described above, is preferably used for a sunscreen because the feeling of use of the product is improved.

As described above, the oil-based particles according to one aspect of the present invention are ensured stability even when a large amount of polar oil such as ultraviolet absorbing polar oil is contained.

The ratio of (A) the polar oil to the total mass of the oil-based particles according to one aspect of the present invention is 10 to 90% by mass or more, preferably 20 to 80% by mass or more, and more preferably 30 to 70% by mass or more.

### <(B) Oily Gelling Agent>

(B) The oily gelling agent forms the oil-based particles and enhances stability. Accordingly, any substance that can enhance the stability of the lipid shell containing a large amount of oil can be used as the oily gelling agent according to one aspect of the present invention. Examples of such an oily gelling agent include, but are not limited to, amino acid derivatives, lime soaps, lipophilic bentonites, dextrin fatty acid esters, sucrose fatty acid esters, benzylidene derivatives of sorbitol, and the like.

Specifically, examples of the amino acid derivatives include acyl amino acid alkylamide, N-lauroyl-L-glutamic acid, α,γ-di-n-butylamine, and the like.

Examples of the lime soaps include aluminum stearate, magnesium stearate, zinc myristate, and the like, in which hydroxyl groups remain.

Examples of the lipophilic bentonites include dimethylbenzyldodecylammonium montmorillonite clay, dimethyldioctadecylammonium montmorillonite clay, and the like.

Examples of the dextrin fatty acid esters include dextrin palmitate, dextrin oleate, dextrin stearate, and the like.

Examples of the sucrose fatty acid esters include those in which 3 or less of the 8 hydroxyl groups are esterified with higher fatty acids, and the higher fatty acids are stearic acid and palmitic acid.

Examples of the benzylidene derivatives of sorbitol include monobenzylidene sorbitol, dibenzylidene sorbitol, and the like.

Preferably, examples include acyl amino acid alkylamides, and specifically, dibutyl lauroyl glutamide and dibutyl ethylhexanoyl glutamide are particularly preferable. The oil-based particles according to one aspect of the present invention may contain these ingredients alone or in combination. When the oily gelling agent is contained, the oil-based particles are stabilized, and the solidifying power of the solid oil is reduced and the hardness can be adjusted so as to be suitable for crushing and stretching the oil particles on the skin.

Preferably, the melting point of (B) the oily gelling agent is 60° C. or more. The oily gelling agent may be contained in an amount of 0.01 to 20% by mass, preferably 0.05 to 15% by mass, and more preferably 0.50 to 10% by mass, in the total mass of the oil-based particles according to one aspect of the present invention.

### <(C) Plate Powder>

(C) Examples of plate powder include talc, mica, mica, silicone powder, titanium oxide powder, zinc oxide powder, iron oxide powder, magnesium oxide powder, barium sulfate powder, aluminum hydroxide powder, alumina, calcium carbonate powder, calcium phosphate powder, boron nitride powder, pearl powder, cellulose powder, corn starch, pearl luster pigment, and the like, which include, for example, silicone resin powder or organic resin powder.

Specifically, examples of the organic resin powder include (meth)acrylate resin powder, polyamide resin powder (nylon powder), polyethylene powder, polystyrene powder, copolymer resin powder of styrene and (meth)acrylic acid, benzoguanamine resin powder, polytetrafluoroethylene powder, cellulose powder, trimethylsilsesquioxane powder, and the like. Examples of commercially available organic resin powder include GANZPEARL (manufactured by Aica Kogyo Co., Ltd.), and the like.

Examples of the silicone resin powder include (vinyl dimethicone/methicone silsesquioxane) crosspolymer, and organopolysiloxane elastomer powder and composite powder using the same as the mother powder. Examples of commercially available spherical silicone resin powder include TREFIL E-505C, TREFIL E-506C, TREFIL E-506S, and TREFIL HP40T (each manufactured by Dow Corning Toray Silicone Co., Ltd.); Tosperl 145A (manufactured by Toshiba Silicone Co., Ltd.); silicone powder KSP-100 and KSP-300 (manufactured by Shin-Etsu Chemical Co., Ltd.); and the like.

These may be contained alone or in combination of two or more. Among them, talc, mica, and silicone powder are preferable. As the silicone powder, (vinyl dimethicone/methicone silsesquioxane) crosspolymer is particularly preferable.

As the plate powder according to the present invention, it is preferable to use one having an average particle size of 1 to 100 μm by a laser diffraction method and a ratio (aspect ratio, a/b) of the long side length (a) to the thickness (b) of 2 to 200.

The plate powder used in the present invention may be subjected to hydrophobic surface treatment. Examples of the hydrophobic surface treatment include, but are not particularly limited to, silicone treatment, fatty acid treatment, fatty acid soap treatment, fatty acid ester treatment, and the like.

Specifically, examples of the silicone treatment include treatment with: silicone oils such as methyl hydrogen polysiloxane, dimethyl polysiloxane, methyl phenyl polysiloxane; alkylsilanes such as methyl trimethoxysilane, ethyltrimethoxysilane, hexyltrimethoxysilane, octyltrimethoxysilane; and a fluoroalkylsilanes such as trifluoromethyl ethyl trimethoxysilane, heptadecafluorodecyl trimethoxysilane; and the like.

Examples of the fatty acid treatment include treatment with: palmitic acid, isostearic acid, stearic acid, lauric acid, myristic acid, behenic acid, oleic acid, rosin acid, 12-hydroxystearic acid, and the like.

Examples of the fatty acid soap treatment include treatment with: aluminum stearate, calcium stearate, 12-hydroxystearic acid, and the like.

Examples of the fatty acid ester treatment include treatment with: dextrin fatty acid ester, cholesterol fatty acid ester, sucrose fatty acid ester, starch fatty acid ester, and the like. These may be contained alone or in combination of two or more. Among these, treatment with dimethyl polysiloxane or calcium stearate is preferable. These hydrophobization treatments may be performed in accordance with conventional procedures. Among these, hydrophobized talc, hydrophobized mica, hydrophobized (vinyl dimethicone/methicone silsesquioxane) crosspolymer, and the like, are particularly preferable.

The content of (C) the plate powder may be 0.1 to 5.0% by mass, and preferably 0.5 to 2.5% by mass, in the total mass of the oil-based particles according to one aspect of the present invention. When the content of (C) the plate powder is within the range, the desired effect of inhibiting aggregation of the oil-based particles is sufficiently exhibited, and the feeling of use of the oil-based particles becomes excellent.

When (B) the oily gelling agent is contained by 0.50 to 10% by mass and (C) the plate powder is contained by 0.5 to 2.5% by mass in the total mass of the oil-based particles according to one aspect of the present invention, formation and stability of the oil-based particles are enhanced.

### <(D) Higher Alcohol>

When the oil-based particles according to one aspect of the present invention contain a higher alcohol as a solid component, stable oil-based particles can be provided. Examples of (D) the higher alcohol include isostearyl alcohol, lauryl alcohol, myristyl alcohol, cetyl alcohol, stearyl alcohol, behenyl alcohol, bacyl alcohol, octyldodecanol, and the like having 16 or more carbon atoms. These may be contained alone or in combination with two or more. In particular, behenyl alcohol and bacyl alcohol are preferably used. When these ingredients are used, stability at high temperature and versatility of the oil-based particles according to one aspect of the present invention are enhanced.

The higher alcohol may be contained by 1 to 80% by mass, preferably 5 to 50% by mass, and more preferably 10 to 20% by mass, in the total mass of the oil-based particles according to one aspect of the present invention.

### <(E) Surfactant>

The oil-based particles according to one aspect of the present invention have a configuration different from that of emulsified particles produced by using a surfactant. Therefore, no surfactant is used to produce the oil-based particles. However, for example, a particle including an emulsified particle inside the lipid shell may be produced. Thus, the oil-based particles according to one aspect of the present invention may also contain a surfactant.

As the surfactant according to one aspect of the present invention, any surfactant may be contained as long as the surfactant forms an emulsified particle included in the oil-based particle according to one aspect of the present invention. Examples of the surfactant include silicone surfactants, fluorine surfactants, polymer surfactants, non-ionic surfactants, cationic surfactants, anionic surfactants, amphoteric surfactants, and the like.

For example, the silicone surfactant is a polymer of an organosilicon compound having a surface-active action, and is highly water-repellent. The silicone surfactant is widely used as a film-forming agent in cosmetics, such as foundation, sunscreen, makeup bases, and conditioning agents for hair treatments. Examples of the silicone surfactant suitable for one aspect of the present invention include, but are not limited to, (PEG-10/lauryl dimethicone) crosspolymer, (PEG-15/lauryl dimethicone) crosspolymer, (dimethicone/(PEG-10/15)) crosspolymer, PEG/PPG-20/22 butyl ether dimethicone, PEG-10 dimethicone, PEG-11 methyl ether dimethicone, PEG-3 dimethicone, PEG-9 dimethicone, PEG-9 methyl ether dimethicone, polyglyceryl-3 disiloxane dimethicone, lauryl PEG-9 polydimethylsiloxyethyl dimethicone, lauryl polyglyceryl-3 polydimethylsiloxyethyl dimethicone, and the like.

The surfactant may be contained by 0.5% or less in the total amount of the oil-based particles according to one aspect of the present invention.

### <(F) (Acrylates/Alkyl Acrylate (C10-30)) Crosspolymer>

The oil-based particles according to one aspect of the present invention may contain a (acrylates/alkyl acrylate (C10-30)) crosspolymer. The (acrylates/alkyl acrylate (C10-30)) crosspolymer is an alkyl-modified carboxyvinyl polymer of a water-soluble polymer, has surface activity, and also has an action of preventing aggregation and coalescence of large particles, and thus is a particularly preferable water-soluble thickener for use in the oil-based particles according to one aspect of the present invention. The oil-based particles that contain the (acrylates/alkyl acrylate (C10-30)) crosspolymer are capable of retaining the form of the oil-based particles according to one aspect of the present invention, maintaining their stability, and are uniformly dispersed in the aqueous solution.

The (acrylates/alkyl acrylate (C10-30)) crosspolymer may be contained by 0.01 to 10% by mass, and preferably 0.04 to 5% by mass, in the total mass of the oil-based particles according to one aspect of the present invention.

<Oil other than (A) Polar Oil>

The oil particle according to one aspect of the present invention may contain oil other than (A) the polar oil. The oil is not particularly limited, and may be an oily ingredient commonly used in external compositions such as cosmetics, quasi-drugs, and the like. The oily ingredient other than the polar oil includes solid oil and liquid oil.

The solid oil is solid or semi-solid oil at room temperature (25° C.). Examples of the solid or semi-solid oil at room temperature (25° C.) include, but are not limited to, hydrocarbon oils, higher fatty acids, higher alcohols, vegetable oils, animal oils, mineral oils, liquid oils, perfumes, aliphatic monocarboxylic acid lanolin alcohol esters, and the like, in addition to (D) the higher alcohol described above.

Specifically, examples of the hydrocarbon oils include solid paraffin, microcrystalline wax, ceresin, beeswax, Bareco wax, polyethylene wax, silicone wax, Vaseline, and the like.

Examples of the higher fatty acids include myristic acid, palmitic acid, stearic acid, behenic acid, 12-hydroxystearic acid, and the like.

Examples of the higher alcohols other than (D) the higher alcohol include straight chain alcohols (for example, behenyl alcohol, stearyl alcohol, cetyl alcohol, bacyl alcohol, and the like) and branched chain alcohols (for example, monostearyl glycerin ether (bacyl alcohol)-2-decyltetradecinol, lanolin alcohol, cholesterol, phytosterol, hexyldodecanol, isostearyl alcohol, octyldodecanol, and the like), and the like.

Examples of the vegetable oils include cocoa butter, hardened castor oil, hardened oil, hydrogenated palm oil, palm oil, hardened palm oil, and the like.

Examples of the animal oils include various hydrogenated animal and vegetable fats and oils, whale oil, beeswax, and the like.

Examples of the waxes and the like include, but are not limited to, carnauba wax, candelilla wax, jojoba wax, lanolin, shellac wax, Japan wax, powdered polyethylene, Vaseline, and the like. Among these, in particular, a solid oil having a melting point of 45° C. to 75° C. is preferable, and a solid oil having a melting point of 50° C. to 70° C. is more preferable. When the melting point is higher than 45° C., the stability of the composition dispersed in the oil-based particles becomes good, and when the melting point is less than 75° C., the preparation of an external composition using the oil-based particles becomes easy. Examples of the solid oil include, but are not limited to, higher alcohols having 16 or more carbon atoms such as cetyl alcohol, stearyl alcohol, behenyl alcohol, and the like, and candelilla wax. Further, straight chain saturated higher alcohols having no unsaturated bond and having 18 or more carbon atoms are more preferable. These ingredients may be contained alone or in combination of two or more.

The content of the solid oil is preferably 10 to 50% by mass, and further preferably 10 to 20% by mass, in the oil phase of the oil-based particles according to one aspect of the present invention. When the content of the solid oil in the oil phase is 10% by mass or more, the stability of the composition dispersed in the oil-based particles becomes good, and when the content is less than 50% by mass, the oil-based particles tend to be easily crushed when applied to the skin and provide a better skin compatibility.

The liquid oil is liquid oil at room temperature (25° C.). Examples of the liquid oil include, but are not limited to, silicone oils, hydrocarbon oils, higher fatty acids, higher alcohols, vegetable oils, animal oils, mineral oils, liquid oils, perfumes, and the like.

Specifically, examples of the silicone oil include, but are not limited to, dimethyl polysiloxanes such as dimethicone, phenyldimethicone, decamethyl tetrasiloxane, dodecamethyl pentasiloxane, tetradecamethyl hexasiloxane, and hexadecamethyl heptasiloxane; cyclic polysiloxanes such as octamethylcyclotetrasiloxane, decamethylcyclopentasiloxane, dodecamethylcyclohexasiloxane, and tetradecamethylcycloheptasiloxane (cyclic dimethyl polysiloxane); methylphenyl polysiloxane, methylhydrogen polysiloxane, decamethyl polysiloxane, dodecamethyl polysiloxane, tetramethyltetrahydrogen polysiloxane, highly polymerized methyl polysiloxane, amino acid modified silicone, caprylyl methicone, and the like.

The silicone oil is used as a major ingredient in an oil phase of oil-based particles. For example, the silicone oil is recognized as a necessary ingredient to improve a feeling when used of products containing oil-based particles for reasons such that when a certain amount of the silicone oil is not contained, a refreshing feeling when used cannot be obtained. However, it is preferable that oil-based particles impart such a feeling of use even when the amount of the polar oil is increased. Among polymers of siloxanes in which a methyl group and the like is bonded to silicon of a silicon-oxygen bond, it is preferable that the cyclic silicone oil, which is a cyclic oil content, is not contained. The oil-based particles according to one aspect of the present invention are highly stable and impart an excellent feeling when used, even when the cyclic silicone is not contained.

Examples of the cyclic silicone oil include, but are not limited to, dimethyl polysiloxanes such as decamethyl tetrasiloxane, dodecamethyl pentasiloxane, tetradecamethyl hexasiloxane, hexadecamethyl heptasiloxane, and the like; and cyclic polysiloxanes (cyclic dimethyl polysiloxanes) such as octamethylcyclotetrasiloxane, decamethylcyclopentasiloxane, dodecamethylcyclohexasiloxane, tetradecamethylcycloheptasiloxane, and the like.

Examples of the hydrocarbon oils include isododecane, isohexadecane, isoparaffin, liquid paraffin, ozocerite, squalane, vegetable squalane, jojoba alcohol, pristane, α-olefin oligomer, polybutene, hydrogenated polyisobutene, and the like.

Examples of the higher fatty acids include glyceryl trioctanoate, glyceryl triisopalmitate, triglyceride, oleic acid, fatty acids from tall oil, isostearic acid, and the like.

Examples of the higher alcohols include straight chain alcohols (for example, lauryl alcohol, isostearyl alcohol, oleyl alcohol, and the like) and branched chain alcohols (for example, octyldodecanol, and the like), and the like.

Examples of the vegetable oils include avocado oil, camellia oil, macadamia nut oil, evening primrose oil, corn oil, olive oil, rapeseed oil, sesame oil, persic oil, wheat germ oil, camellia kissi seed oil, castor oil, linseed oil, safflower oil, cottonseed oil, perilla oil, soybean oil, peanut oil, tea seed oil, kaya oil, rice bran oil, palm kernel oil, tung oil, jojoba oil, sunflower oil, almond oil, meadowfoam oil, germ oil, and the like.

Examples of the animal oils include squalane, turtle oil, mink oil, egg yolk oil, and the like.

Further examples of the liquid oil include, but are not limited to, isocetyl isostearate, ethylene glycol di-2-ethylhexanoate, cetyl 2-ethylhexanoate, trimethylol propane tri-2-ethyl hexanoate, pentaerythritol tetra-2-ethyl hexanoate, cetyl octanoate, pentaerythrityl tetraoctanoate, glyceride tri-2-heptyl undecanoate, diisobutyl adipate, 2-hexyldecyl sebacate, glyceryl trioctanoate, glyceryl triisopalmitate, diisostearyl malate, isopropyl myristate, 2-octyldodecyl oleate, hexyldecyl dimethyl octanoate, 2-hexyldecyl myristate, octyldodecyl myristate, isopropyl palmitate, 2-hexyldecyl palmitate, 2-heptylundecyl palmitate, butyl stearate, isocetyl stearate, decyl oleate, dodecyl oleate, oleyl oleate, myristyl lactate, cetyl lactate, cholesteryl 12-hydroxystearate, castic oil fatty acid methyl ester, 2-ethylhexyl succinate, 2-hexyldecyl adipate, di(2-heptylundecyl) adipate, diisopropyl sebacate, di(2-ethylhexyl) sebacate, neopentyl glycol dicaprate, neopentyl glycol dioctanoate, glyceryl tri(2-ethylhexanoate), glyceryl trimyristate, tri 2-heptylundecanoate glyceride, trimethylolpropane triisostearate, glyceryl tri(2-ethylhexanoate), isopropyl myristate, isopropyl palmitate, hexyl laurate, oleyl oleate, decyl oleate, octyldodecyl myristate, hexyldecyl dimethyl octanoate, diethyl phthalate, dibutyl phthalate, dioctyl succinate, fluorine-modified oils, trioctanoin, tripropylene glycol dipivalate, and the like. These ingredients may be contained, as the oil according to one aspect of the present invention, alone or in combination of two or more.

The oil may be contained by 1 to 90% by mass, preferably 10 to 85% by mass, and even more preferably 15 to 80% by mass, in the total mass of the oil-based particles according to one aspect of the present invention. In the oil phase of the oil-based particles, the oil may be contained by 50 to 90% by mass, preferably 80 to 90% by mass. When the oil is contained by 50% by mass or more, a better skin compatibility when applied to the skin can be provided, and when the oil is contained by 90% by mass or less, good stability of the dispersed composition in the oil-based particles can be obtained. As other ingredients, an ultraviolet absorber that is not the polar oil may be included.

The content of the oil other than (A) the polar oil containing the solid oil and the liquid oil varies depending on applications and desired feelings when used, and is not particularly limited, but is preferably 1 to 20% by mass, and more preferably 3 to 10% by mass, in the total mass of the oil-based particles according to one aspect of the present invention. When the content of the oil phase is 1% by mass or more, a sufficient moist feeling when applied to the skin as an external agent can be provided, and when the content is 20% by mass or less, a good feeling when used can be obtained.

### <Method for Producing Oil-Based Particle>

The oil-based particles according to an aspect of the present invention may be produced by publicly-known methods. For example, the oil-based particles may be produced as follows. An oil phase containing solid oil and liquid oil is added, at a temperature above the melting point of the solid oil, into an aqueous phase at a temperature above the melting point of the solid oil containing a water-soluble thickener, and the mixture is sheared and mixed until it reaches a temperature equal to or lower than the melting point of the solid oil.

Typically, an oil phase is introduced into an aqueous phase by injecting a mixture of oily ingredients liquified by heating to a temperature of around 70° C. directly from the bottom of the aqueous phase into the aqueous phase at a similar temperature, using an injection means such as a liquid feed pump and the like.

Then, for example, by shear mixing until the temperature reaches 40° C. to 50° C., which is equal to or lower than the melting point of the solid oil, oil particles that have been formed so that the solid oil crystallizes to include other oily ingredients are crushed by shear force, and a fine dispersion of the oil is formed. As a result, the oil phase containing the solid oil can be stably dispersed in the aqueous phase, preferably as a particle having an average maximum size of a primary particle of 0.1 to 30 μm, more preferably 1 to 15 μm, without causing floating substances or agglomerates of the oily ingredients. The shear mixing may be performed while the oil phase is being added to the aqueous phase, or the shear mixing may be started immediately after the addition is completed without performing the shear mixing during the addition. The shear mixing may be performed with the mixture being cooled using, for example, ice and the like, or with the mixture being cooled naturally at room temperature. As used herein, “shear mixing until the temperature reaches a temperature equal to or lower than the melting point of the solid oil” means that the shear mixing is started from a temperature higher than the melting point of the solid oil, and the shear mixing is performed continuously or intermittently until the temperature of the mixture of the oil phase and the aqueous phase becomes a temperature equal to or lower than the melting point of the solid oil. The method for producing according to the present invention includes, for example, the case where the shear mixing is performed until the temperature reaches a temperature near the melting point of the solid oil, the shear mixing is then stopped, and the shear mixing is performed again at a temperature equal to or lower than the melting point of the solid oil.

The shear mixing may be performed using any device that can perform mixing under shear force. For example, the shear mixing may be performed using a high-speed shear disperser such as a homomixer, a homodisper, a flow jet mixer, an ultramixer, a colloidal mill, and the like, or a high-pressure shear disperser such as a homogenizer and the like. The shear mixing may be performed using any other device as long as the object of the present invention is achieved. Among these, by performing the shear mixing using a homomixer, a more homogeneous and stable oil fine dispersed composition can be produced. The rotational speed when using the high-speed shear disperser, or the pressure when using the high-pressure shear disperser, depends on the type and power of the device used, and is not particularly limited. For example, the rotational speed when using the high-speed shear disperser, such as a homomixer, is preferably 500 to 15,000 rpm, more preferably 1,000 to 10,000 rpm, and even more preferably 4,000 to 9,000 rpm. When the rotational speed of the high-speed shear disperser is less than 500 rpm, oil particles of the expected size may not be obtained, and when the rotational speed is more than 10,000 rpm and further increased, the effect of micronization cannot be further improved. The pressure when using the high-pressure shear disperser, such as a homogenizer, is preferably 50 to 1000 kgf/cm², more preferably 100 to 500 kgf/cm², and even more preferably 200 to 400 kgf/cm². When the pressure of the high-pressure shear disperser is less than 50 kgf/cm², oil particles of the expected size may not be obtained, and when the pressure is more than 1000 kgf/cm² and further increased, the effect of micronization cannot be further improved.

The duration of the shear mixing varies depending on the time required to bring the temperature of the mixture of the oil phase and the liquid phase to a temperature equal to or lower than the melting point of the solid oil, and is not particularly limited, but preferably 1 to 60 minutes, more preferably 2 to 30 minutes, and even more preferably 3 to 10 minutes.

After the dispersion process by the shear mixing, it is preferable to perform stirring of the dispersion to increase homogeneity and stability. Typically, the stirring may be performed using a slow stirrer such as a propeller mixer, a paddle mixer, an anchor mixer, a gate mixer, a planetary mixer, and the like, at a relatively slow rotational speed, such as, for example, 10 to 1500 rpm, and more preferably 20 to 300 rpm, for, for example, 30 minutes to 10 hours, and more preferably 1 hour to 4 hours. In this manner, the desired oil-based particles can be produced, but the method for producing the oil-based particles is not limited thereto.

### <External Composition>

Another aspect of the present invention include an external composition in which the oil-based particles according to one aspect of the present invention are dispersed in an aqueous solvent. Such an external composition can be used, for example, as cosmetics or skin external agents that can be used by application or the like on the outer skin of humans or animals. The cosmetics are not particularly limited and include, for example, cosmetics for skin care (also referred to as basic cosmetics), such as lotion, serum, emulsion, cream, liquid facial cleanser, and the like; cosmetics for makeup (also referred to as make-up cosmetics), such as foundation, blusher, lips rouge, gloss, eyebrow cosmetics, eyeliners, eye shadow, mascara, manicure, perfume, and the like; and cosmetics for scalp hair (also referred to as scalp hair cosmetics), such as shampoo, hair conditioner, hair treatment, hair essence, hair mist, scalp lotion, scalp cream, hair tonic, a foam-type hair growth agent, a liquid-type hair growth agent, shampoo, hair conditioner, hair treatment, hair essence, hair mist, scalp lotion, scalp cream, hair tonic, a foam-type hair growth agent, a liquid-type hair growth agent, and the like. In the present embodiment, preferably, the cosmetics are makeup bases, sunscreen, and scalp hair cosmetics.

The external composition according to one aspect of the present invention may also be provided, by utilizing the characteristics of the lipid shell, as a product in a form in which fine oil-based particles are recognized in the aqueous phase, so to speak, a form in which “emulsified particles can be seen”.

Examples of the aqueous solvent include water or a solvent that is identified with water with respect to the oil-based particles, which can maintain the form of the oil-based particles according to one aspect of the present invention and maintain the stability thereof. Specifically, examples of the aqueous solvent include an aqueous solution containing a lower alcohol, a polyhydric alcohol, a preservative, a pH adjusting agent, a sequestering agent for metal ions, a neutralizing agent, an antioxidant, a dye, and the like.

The oil-based particles according to one aspect of the present invention may be used as an element to be added in a production process of a typical external composition to provide an external composition having various dosage forms and shapes. That is, the oil-based particles may be used for any external composition of any dosage form, such as solution type, solubilization type, emulsion type, powder dispersion type, water-powder two-phase type, water-oil-powder three-phase type, and the like.

The external composition according to one aspect of the present invention may take any form of product, and may be used as an external composition for facial, body, or scalp hair, such as, for example, lotion, emulsion, cream, facial mask, and the like.

### <Other Ingredients>

The oil-based particles according to one aspect of the present invention may contain water-soluble or oil-soluble ingredients in addition to the ingredients described above, unless departing from the spirit of the present invention. Examples of such ingredients in the aqueous phase include lower alcohols such as ethanol and other aqueous solvents, in addition to typical aqueous moisturizers such as glycerin, dipropylene glycol, 1,3-butylene glycol, and the like. If necessary, other water-soluble ingredients may be contained such as, for example, arbutin, ascorbic acid and its derivatives, tranexamic acid and its derivatives, and the like. Further, examples include water-soluble ultraviolet absorbers, ultraviolet scattering agents, whitening agents, thickeners, antioxidants, antimicrobial preservatives, chelating agents, vitamins, hormones, natural extracts extracted with aqueous alcohol, pH adjusting agents, preservatives, dyes, perfumes, and the like.

In particular, a water-soluble thickener may be contained in the aqueous phase. The water-soluble thickener increases the viscosity of the composition and helps the oil particles to stably disperse in the aqueous phase. Also, the water-soluble thickener has a function of adjusting the viscosity of the composition to a viscosity suitable for use. The water-soluble thickener is not particularly limited as long as it can thicken the aqueous solvent, and examples thereof include water-soluble polymers capable of thickening the aqueous solvent. Examples of such water-soluble polymers include plant-based polymers, microbial-based polymers, animal-based polymers, cellulose-based polymers, alginic acid-based polymers, vinyl-based polymers, acrylic-based polymers, and inorganic-based water-soluble polymers.

Examples of the water-soluble polymers other than (F) the (acrylates/alkyl acrylate (C10-30)) crosspolymer described above include, specifically, as the plant-based polymers, gum arabic, tragacanth gum, galactan, carob gum, guar gum, gum karaya, carrageenan, pectin, agar, Pyrus cydonia seed (Pyrus cydonia), algae colloid (extract of Phaeophyta), and the like.

Examples of the microbial-based polymers include dextran, succinoglycan, pullulan, xanthan gum, and the like.

Examples of the animal-based polymers include collagen, casein, albumin, gelatin, and the like.

Examples of the cellulose-based polymers include methyl cellulose, nitrocellulose, ethyl cellulose, methyl hydroxypropyl cellulose, hydroxy ethyl cellulose, sodium cellulose sulfate, hydroxypropyl cellulose, carboxymethyl cellulose sodium, crystalline cellulose, cellulose powder, and the like.

Examples of the alginic acid-based polymers include sodium alginate, propylene glycol alginate, and the like.

Examples of the vinyl-based polymers include polyvinyl alcohol, polyvinyl methyl ether, polyvinylpyrrolidone, carboxy vinyl polymers, alkyl-modified carboxy vinyl polymers, and the like.

Examples of the acrylic-based polymers include polyoxyethylene-based polymers, polyoxyethylene/polyoxypropylene copolymer-based polymers, sodium polyacrylate, polyethyl acrylate, and polyacrylamide.

Examples of the inorganic-based water-soluble polymers include polyethyleneimine, cationic polymers, bentonite, aluminum magnesium silicate, laponite, hectorite, silicic anhydride, and the like.

When agar is used as the water-soluble thickener other than the (F) (acrylates/alkyl acrylate (C10-30)) crosspolymer described above, a new feeling when used can be obtained that is not provided by other water-soluble thickeners. A thickener made of a microgel as described in Japanese Laid-Open Patent Application No. 2004-43785 may be used in the present invention.

In the oil phase, sterols, ultraviolet absorbers such as octyl paramethoxycinnamate, 2-hydroxy-4-methoxybenzophenone, and the like, ultraviolet scattering agents, whitening agents, moisturizers, thickeners, antioxidants, antimicrobial preservatives, chelating agents, vitamins such as vitamin A and its derivatives, vitamin D and its derivatives, vitamin E and its derivatives, vitamin K and its derivatives, and the like, hormones, arginine, amino acids, anti-inflammatory agents, antimicrobial agents, astringents, refreshing agents, sterols, extracts derived from animals, plants, or microorganisms, alcohols, polymeric substances, neutralizing agents, pH adjusting agents, oil-soluble agents, dyes, perfumes, and the like may be contained. These substances may be contained in the oil phase, or the surface of the crystal of the water-soluble agent such as vitamin C, arbutin, and the like that has been hydrophobized may be dispersed in the oil phase. In addition, the oil-soluble agents may be contained in the oil phase.

Specifically, the oil-based particles according to one aspect of the present invention may contain the following.

For example, in addition to the above-described ultraviolet polar oil as the polar oil, any commonly used ultraviolet absorber may be used. Specific examples include sodium hydroxymethoxybenzophenone sulfonate, sodium dihydroxydimethoxybenzophenone disulfonate, hydroxymethoxybenzophenone sulfonic acid and its trihydrate, phenylbenzimidazole sulfonic acid, ferulic acid, terephthalylidene dicamphor sulfonic acid, benzylidene camphor sulfonic acid, and the like. In particular, it is preferable to contain in combination of sodium hydroxymethoxybenzophenone sulfonate and a ultraviolet absorbing polar oil such as ethylhexyl methoxycinnamate, bis-ethylhexyloxyphenol methoxyphenyltriazine, and diethylaminohydroxybenzoyl hexyl benzoate.

When the oil-based particles according to one aspect of the present invention are used as sunscreen, an ultraviolet scattering agent may also be contained in addition to the above-described ultraviolet absorber. As the ultraviolet scattering agent, any powder particles having an ultraviolet scattering effect may be used. For example, particulate titanium oxide, particulate zinc oxide, and the like may be used. Specific examples include, but are not limited to, in addition to titanium oxide, other non-limiting examples include octyltriethoxysilane-treated particulate zinc oxide and dextrin palmitate-treated particulate zinc oxide.

Examples of the whitening agents include tranexamic acid, potassium 4-methoxysalicylate, nicotinic acid amide, and the like.

Examples of the moisturizers include glycerin, 1,3-butylene glycol, polyethylene glycol, sorbitol, xylitol, maltitol, and the like.

Examples of the thickener include plant-based polymers (for example, gum arabic, tragacanth gum, galactan, guar gum, carrageenan, and the like), microbial polymers (for example, xanthan gum, dextran, succinoglycan, pullulan, and the like), animal-based polymers (for example, collagen, casein, albumin, gelatin, and the like), starch-based polymers (for example, carboxymethyl starch, methyl hydroxypropyl starch, and the like), and alginic acid-based polymers (for example, sodium alginate, propylene glycol alginate, and the like). Examples also include a synthetic crosspolymer such as dimethylacrylamide/sodium acryloyldimethyl taurate crosspolymer. In particular, (ammonium acryloyl dimethyltaurate/VP) copolymer and ingredients similar to (F) the (acrylates/alkyl acrylate (C10-30)) crosspolymer are preferable.

Examples of the antioxidants include butyl hydroxytoluene, tocopherols, phytic acid, ascorbic acid, sodium pyrosulfite, and the like.

Examples of the antimicrobial preservatives include benzoic acid, sodium benzoate, salicylic acid, sorbic acid, alkylesters of parahydroxybenzoic acid (ethyl paraben, butyl paraben, and the like), methyl paraben, sodium dehydroacetate, phenoxyethanol, hexachlorophene, and the like.

Examples of the chelating agents include salicylic acid, edetic acid, metaphosphoric acid, and the like.

Examples of the vitamins include: vitamins such as vitamin A and its derivatives; vitamin B such as vitamin B6 hydrochloride, vitamin B6 tripalmitate, vitamin B6 dioctanoate, vitamin B2 and its derivatives, vitamin B12, vitamin B15 and its derivatives, and the like; vitamin C such as ascorbic acid, ascorbic acid sulfate ester (salt), ascorbic acid phosphate ester (salt), ascorbic acid dipalmitate, and the like; vitamins E such as α-tocopherol, β-tocopherol, γ-tocopherol, vitamin E-acetate, vitamin E-nicotinate, and the like; vitamin D; vitamin H; pantothenic acid; and pantothetine; nicotinic acid amide, benzyl nicotinate, γ-olizanol, allantoin, glycyrrhizic acid (salt), glycyrrhetinic acid and its derivatives, hinokitiol, mucidin, bisabolol, eucalyptol, thymol inositol, pantothenyl ethylether, ethinyl estradiol, cepharanthine, placenta extract, and the like.

Examples of the alcohols include ethanol, propanol, isopropanol, isobutyl alcohol, t-butyl alcohol, and the like.

Examples of the polymer substances include: plant-based polymers such as polyhydric alcohol, gum arabic, tragacanth gum, galactan, guar gum, carob gum, gum karaya, gellan gum, and carrageenan; microbial polymers such as xanthan gum, dextran, succinoglycan, pullulan, and sodium biohyaluronate; animal-based polymers such as collagen, casein, and gelatin; starch-based polymers such as carboxymethyl starch, and methyl hydroxypropyl starch; cellulose-based polymers such as methyl cellulose, ethyl cellulose, methyl hydroxypropyl cellulose, hydroxy ethyl cellulose, sodium cellulose sulfate, hydroxypropyl cellulose, carboxymethyl cellulose sodium, and crystalline cellulose; alginic acid-based polymers such as sodium alginate, and propylene glycol alginate; vinyl-based polymers such as polyvinyl alcohol, polyvinyl acetate, polyvinyl methyl ether, polyvinylpyrrolidone, copolymers of vinylpyrrolidone and vinyl acetate, and carboxy vinyl polymers; acrylic-based polymers such as sodium polyacrylate, polyethyl acrylate, alkanolamine polyacrylate, copolymers of alkyl methacrylate and dimethylamino ethyl methacrylate, poly 2-acrylamido-2-methylpropanesulfonic acid, and polymethacryloyloxytrimethyl ammonium; and polymers including 2-methacryloyloxyethyl phosphorylcholine as a monomer.

Examples of other ingredients further include acyl sarcosinate such as sodium lauroylsarcosinate, glutathione; α-hydroxy acids such as aminocaproic acid, citric acid, malic acid, lactic acid, glycolic acid, and tartaric acid, and their salts such as sodium and potassium, Rumex japonicus, Sophora flavescens, Nuphar japonica, orange, sage, yarrow, mallow, Cnidium, Swertia japonica, thyme, Angelica acutiloba, spruce, birch, field horsetail, sponge gourd, horse-chestnut, saxifrage, blackberry lily, chamomile, arnica, lily, mugwort, Chinese peony, aloe, gardenia, Chamaecyparis pisifera, royal jelly, placental extract, pantothenyl ethyl ether, dipotassium glycyrrhizinate, biotin, pyridoxine hydrochloride, adenosine triphosphate, α-lipoic acid, 3-O-ethylascorbic acid, magnesium ascorbyl phosphate, albutin, glucosyl hesperidin, glucosylrutin, and the like.

These substances may be contained within a range that does not impair the effects of the present invention.

### EXAMPLE

The following are examples of the oil-based particles according to one aspect of the present invention. Those skilled in the art will understand that the substances, formulations, ratios, methods, and the like used in the present example can be appropriately changed without departing from the spirit of the present invention. Also, the description of the present example is not intended to limit the present invention to the scope of the example. In the following examples and the like, “%” regarding the content represents “% by mass” unless otherwise specified.

### Examples 1 to 8 and Comparative Examples 1 and 2

Oil-based particles having the compositions described in the following tables were prepared according to conventional procedures. In the following examples, the combination of the proportions of the oily gelling agent and the polar oil so as to form oil-based particles was examined.

Specifically, as an evaluation method, the oil-based particles were prepared, subjected to follow-up observation at 25° C. for 2 weeks, and evaluated as follows.

A: The form of the oil-based particles did not change.
B: About 20% of the oil-based particles aggregated (coalesced).
C: About 20 to 50% of the oil-based particles aggregated (coalesced).
D: 50% or more of the oil-based particles aggregated (coalesced), or no oil-based particle was formed at the time of preparation.

The results are described in Tables 1 and 2 below.

TABLE 1

| INGREDIENT NAME | EXAMPLE 1 | EXAMPLE 2 | EXAMPLE 3 | EXAMPLE 4 |
|---|---|---|---|---|
| ION-EXCHANGED WATER | BALANCE | BALANCE | BALANCE | BALANCE |
| ETHANOL | 10 | 10 | 10 | 10 |
| (AMMONIUM ACRYLOYL | 0.3 | 0.3 | 0.3 | 0.3 |
| DIMETHYLTAURATE/VP) | | | | |
| COPOLYMER | | | | |
| (ACRYLATES/ALKYL ACRYLATE | 0.02 | 0.02 | 0.01 | 0.01 |
| (C10-30)) CROSSPOLYMER | | | | |
| PHENYLDIMETHICONE | 3.75 | 3.75 | 3.75 | 3.75 |
| DIMETHICONE | 2 | 2 | 4 | 4 |
| DIISOPROPYL SEBACATE | 5 | 5 | — | — |
| BEHENYL ALCOHOL | 2.2 | 2.2 | 1.5 | 1.5 |
| BACYL ALCOHOL | 0.75 | 0.75 | 0.5 | 0.5 |
| DIBUTYL LAUROYL | 0.2 | — | 0.75 | 0.25 |
| GLUTAMIDE | | | | |
| DIBUTYL ETHYLHEXANOYL | — | 0.2 | — | — |
| GLUTAMIDE | | | | |
| ETHYLHEXYL | 7.5 | 7.5 | 4 | 4 |
| METHOXYCINNAMATE | | | | |
| BIS-ETHYLHEXYLOXYPHENOL | 2 | 2 | — | — |
| METHOXYPHENYLTRIAZINE | | | | |
| SODIUM | 2 | 2 | — | — |
| HYDROXYMETHOXYBENZOPHENONE | | | | |
| SULFONATE | | | | |
| DIETHYLAMINO | 2 | 2 | — | — |
| HYDROXYBENZOYL HEXYL | | | | |
| BENZOATE | | | | |
| (VINYL DIMETHICONE/METHICONE | 2 | 2 | — | — |
| SILSESQUIOXANE) CROSSPOLYMER | | | | |
| HYDROPHOBIZED TALC | 0.19 | 0.19 | 0.1 | 0.1 |
| HYDROPHOBIZED SYNTHETIC | — | — | — | — |
| PHLOGOPITE | | | | |
| LIPID SHELL STABILITY | A | A | A | A |
| INGREDIENT NAME | EXAMPLE 5 | EXAMPLE 6 | EXAMPLE 7 | EXAMPLE 8 |
| ION-EXCHANGED WATER | BALANCE | BALANCE | BALANCE | BALANCE |
| ETHANOL | 10 | 10 | 10 | 10 |
| (AMMONIUM ACRYLOYL | 0.3 | 0.3 | 0.3 | 0.3 |
| DIMETHYLTAURATE/VP) | | | | |
| COPOLYMER | | | | |
| (ACRYLATES/ALKYL ACRYLATE | 0.01 | 0.02 | 0.02 | 0.02 |
| (C10-30)) CROSSPOLYMER | | | | |
| PHENYLDIMETHICONE | 3.75 | 3.75 | — | 3.75 |
| DIMETHICONE | 7.5 | 2 | 4 | 2 |
| DIISOPROPYL SEBACATE | — | 5 | 5 | 5 |
| BEHENYL ALCOHOL | 2.8 | 2.2 | — | 2.2 |
| BACYL ALCOHOL | 0.94 | 0.75 | 1.5 | 0.75 |
| DIBUTYL LAUROYL | 0.47 | 0.2 | — | 0.2 |
| GLUTAMIDE | | | | |
| DIBUTYL ETHYLHEXANOYL | — | — | 0.2 | |
| GLUTAMIDE | | | | |
| ETHYLHEXYL | 4 | 7.5 | 4 | 7.5 |
| METHOXYCINNAMATE | | | | |
| BIS-ETHYLHEXYLOXYPHENOL | 3 | 2 | — | 2 |
| METHOXYPHENYLTRIAZINE | | | | |
| SODIUM | 2 | 2 | — | 2 |
| HYDROXYMETHOXYBENZOPHENONE | | | | |
| SULFONATE | | | | |
| DIETHYLAMINO | — | 2 | — | 2 |
| HYDROXYBENZOYL HEXYL | | | | |
| BENZOATE | | | | |
| (VINYL DIMETHICONE/METHICONE | — | 2 | — | 2 |
| SILSESQUIOXANE) CROSSPOLYMER | | | | |
| HYDROPHOBIZED TALC | 0.19 | — | — | |
| HYDROPHOBIZED SYNTHETIC | — | 0.19 | 0.19 | — |
| PHLOGOPITE | | | | |
| LIPID SHELL STABILITY | A | A | A | B |

TABLE 2

| | COMPARATIVE | COMPARATIVE |
|---|---|---|
| INGREDIENT NAME | EXAMPLE 1 | EXAMPLE 2 |
| ION-EXCHANGED WATER | BALANCE | BALANCE |
| ETHANOL | 10 | 10 |
| (AMMONIUM ACRYLOYL | 0.3 | 0.3 |
| DIMETHYLTAURATE/VP) | | |
| COPOLYMER | | |
| (ACRYLATES/ALKYL | 0.01 | 0.01 |
| ACRYLATE (C10-30)) | | |
| CROSSPOLYMER | | |
| PHENYLDIMETHICONE | 3.75 | 3.75 |
| DIMETHICONE | 4 | 4 |
| BEHENYL ALCOHOL | 1.5 | 1.5 |
| BACYL ALCOHOL | 0.5 | 0.5 |
| ETHYLHEXYL | 4 | 4 |
| METHOXYCINNAMATE | | |
| HYDROPHOBIZED TALC | 0.1 | — |
| LIPID SHELL STABILITY | D | D |

The following table describes formulation examples in which a whitening agent is contained in the oil-based particles according to one aspect of the present invention. As the whitening agent, tranexamic acid, potassium 4-methoxysalicylate, and nicotinic acid amide were used.

TABLE 3

| | FORMULATION | FORMULATION | FORMULATION | FORMULATION | FORMULATION |
|---|---|---|---|---|---|
| INGREDIENT NAME | EXAMPLE 1 | EXAMPLE 2 | EXAMPLE 3 | EXAMPLE 4 | EXAMPLE 5 |
| ION-EXCHANGED WATER | BALANCE | BALANCE | BALANCE | BALANCE | BALANCE |
| TRANEXAMIC ACID | 2 | — | — | — | — |
| POTASSIUM | — | 1 | — | — | — |
| 4-METHOXYSALICYLATE | | | | | |
| NICOTINIC ACID AMIDE | — | — | 5 | — | — |
| ETHANOL | 10 | 10 | 10 | 10 | 10 |
| (AMMONIUM ACRYLOYL | 0.3 | 0.3 | 0.3 | 0.3 | 0.3 |
| DIMETHYLTAURATE/VP) | | | | | |
| COPOLYMER | | | | | |
| (ACRYLATES/ALKYL ACRYLATE | 0.02 | 0.02 | 0.02 | 0.01 | 0.01 |
| (C10-30)) CROSSPOLYMER | | | | | |
| PHENYLDIMETHICONE | 3.75 | 3.75 | 3.75 | 3.75 | 3.75 |
| DIMETHICONE | 2 | 2 | 2 | 7.5 | 7.5 |
| DIISOPROPYL SEBACATE | 5 | 5 | 5 | 1 | 1 |
| BEHENYL ALCOHOL | 2.2 | 2.2 | 2.2 | 2.8 | 2.8 |
| BACYL ALCOHOL | 0.75 | 0.75 | 0.75 | 0.94 | 0.94 |
| DIBUTYL LAUROYL GLUTAMIDE | 0.2 | 0.2 | 0.2 | 0.47 | 0.47 |
| ETHYLHEXYL | 7.5 | 7.5 | 7.5 | — | — |
| METHOXYCINNAMATE | | | | | |
| BIS-ETHYLHEXYLOXYPHENOL | 2 | 2 | 2 | 3 | 3 |
| METHOXYPHENYLTRIAZINE | | | | | |
| SODIUM | 2 | 2 | 2 | — | — |
| HYDROXYMETHOXYBENZOPHENONE | | | | | |
| SULFONATE | | | | | |
| DIETHYLAMINO | 2 | 2 | 2 | 1 | — |
| HYDROXYBENZOYL HEXYL | | | | | |
| BENZOATE | | | | | |
| OCTOCRYLENE | — | — | — | — | 2 |
| ETHYLHEXYL SALICYLATE | — | — | — | 4 | 2 |
| HOMOSALATE | — | — | — | 2 | 2 |
| (VINYL DIMETHICONE/METHICONE | 2 | 2 | 2 | — | — |
| SILSESQUIOXANE) CROSSPOLYMER | | | | | |
| HYDROPHOBIZED TALC | 0.19 | 0.19 | 0.19 | 0.19 | 0.19 |
| HYDROPHOBIZED SILICA | — | — | — | — | — |

| | FORMULATION | FORMULATION | FORMULATION | FORMULATION |
|---|---|---|---|---|
| INGREDIENT NAME | EXAMPLE 6 | EXAMPLE 7 | EXAMPLE 8 | EXAMPLE 9 |
| ION-EXCHANGED WATER | BALANCE | BALANCE | BALANCE | BALANCE |
| TRANEXAMIC ACID | — | 2 | — | — |
| POTASSIUM | — | — | 1 | — |
| 4-METHOXYSALICYLATE | | | | |
| NICOTINIC ACID AMIDE | — | — | — | 5 |
| ETHANOL | 10 | 10 | 10 | 10 |
| (AMMONIUM ACRYLOYL | 0.3 | 0.3 | 0.3 | 0.3 |
| DIMETHYLTAURATE/VP) | | | | |
| COPOLYMER | | | | |
| (ACRYLATES/ALKYL ACRYLATE | 0.01 | 0.01 | 0.01 | 0.01 |
| (C10-30)) CROSSPOLYMER | | | | |
| PHENYLDIMETHICONE | 3.75 | 3.75 | 3.75 | 3.75 |
| DIMETHICONE | 7.5 | 7.5 | 7.5 | 7.5 |
| DIISOPROPYL SEBACATE | 1 | 1 | 1 | 1 |
| BEHENYL ALCOHOL | 2.8 | 2.8 | 2.8 | 2.8 |
| BACYL ALCOHOL | 0.94 | 0.94 | 0.94 | 0.94 |
| DIBUTYL LAUROYL GLUTAMIDE | 0.47 | 0.47 | 0.47 | 0.47 |
| ETHYLHEXYL | — | — | — | — |
| METHOXYCINNAMATE | | | | |
| BIS-ETHYLHEXYLOXYPHENOL | 3 | 3 | 3 | 3 |
| METHOXYPHENYLTRIAZINE | | | | |
| SODIUM | — | — | — | — |
| HYDROXYMETHOXYBENZOPHENONE | | | | |
| SULFONATE | | | | |
| DIETHYLAMINO | 1 | 1 | 1 | 1 |
| HYDROXYBENZOYL HEXYL | | | | |
| BENZOATE | | | | |
| OCTOCRYLENE | — | — | — | — |
| ETHYLHEXYL SALICYLATE | 4 | 4 | 4 | 4 |
| HOMOSALATE | 2 | 2 | 2 | 2 |
| (VINYL DIMETHICONE/METHICONE | — | — | — | — |
| SILSESQUIOXANE) CROSSPOLYMER | | | | |
| HYDROPHOBIZED TALC | 0.19 | 0.19 | 0.19 | 0.19 |
| HYDROPHOBIZED SILICA | 0.05 | 0.05 | 0.05 | 0.05 |

### Example 9

The oil-based particles described in Example 1 were used to prepare a composition dispersed in an aqueous solvent. Subsequently, the oil-based particles were removed from the aqueous solvent and washed with water. The oil-based particles were then dried and a granular external composition was obtained.

The present application claims the priority to Japanese Patent Application No. 2019-239664, filed on Dec. 27, 2019, with the Japanese Patent Office, the entire contents of which are hereby incorporated by reference.

1. Oil-based particles having an average particle size of 0.05 mm to 10 mm, the oil-based particles comprising:
(A) polar oil; and
(B) an oily gelling agent having a melting point of 60° C. or more.

2. The oil-based particles according to claim 1, wherein the polar oil contains ultraviolet absorbing polar oil.

3. The oil-based particles according to claim 1, wherein the polar oil is 30% by mass or more.

4. The oil-based particles according to claim 1, wherein the oily gelling agent is acyl amino acid alkylamide.

5. The oil-based particles according to claim 4, wherein the acyl amino acid alkylamide is at least one selected from a group consisting of dibutyl lauroyl glutamide and dibutyl ethylhexanoyl glutamide.

6. The oil-based particles according to claim 1, further comprising plate powder, higher alcohol, or both.

7. The oil-based particles according to claim 6, wherein the plate powder is talc, mica, or both.

8. The oil-based particles according to claim 1, further comprising a surfactant, wherein a content of the surfactant is 0.5% or less.

9. The oil-based particles according to claim 1, further comprising a (acrylates/alkyl acrylate (C10-30)) crosspolymer.

10. An external composition wherein the oil-based particles according to claim 1 are dispersed in an aqueous solvent.
